(19) United States
(12) Patent Application Publication
(10) Pub. No.: US 2019/0038243 A1
(43) Pub. Date: Feb. 7, 2019

(54) Scan-Based Arterial Input Function for Medical Imaging

(71) Applicant: Siemens Medical Solutions USA, Inc., Malvern, PA (US)

(72) Inventors: Vijay Shah, Knoxville, TN (US); Fei Gao, Knoxville, TN (US); Sven Zuehlsdorff, Knoxville, TN (US)

(21) Appl. No.: 16/007,109

(22) Filed: Jun. 13, 2018

Related U.S. Application Data
(60) Provisional application No. 62/539,566, filed on Aug. 1, 2017.

(51) Int. Cl. A61B 6/00; A61B 6/03; A61B 6/04; A61K 51/00

(52) U.S. Cl. CPC A61B 6/4057; A61B 6/037; A61K 51/00; A61B 6/504; A61B 6/5217; A61B 6/0407

(57) ABSTRACT

A method for operating a medical scanner comprises: injecting a radiopharmaceutical into a patient; performing one or more scans of the patient after the injecting using a medical scanner, each of the one or more scans including sampling an arterial input function of the patient at two or more locations in a same blood vessel of the patient at respectively different times for each location; and estimating the arterial input function of the patient based on the sampling, for use in medical imaging.

This application claims the benefit of U.S. Provisional Patent Application No. 62/539,566, filed Aug. 1, 2017, which is incorporated herein by reference in its entirety.

### FIELD

This disclosure relates generally to medical scanning, and more particularly to methods and systems for determining arterial input function.

### BACKGROUND

Medical imaging, such as X-ray, CT (computed tomography), MR (magnetic resonance imaging), PET (positron emission tomography), and the like have become important clinical tools for evaluation of organ function. One such functional parameter is the perfusion, which characterizes the passage of blood through the vessels of an organ (e.g., heart, brain). Such evaluation procedures are non-invasive or minimally invasive, and may measure the cerebral perfusion by a variety of hemodynamic measurements such as cerebral blood volume (CBV), cerebral blood flow (CBF) and mean transit time (MTT).

The measurement technique may include the administration of contrast agents (which may also be called “tracers”), the tracers being selected as appropriate for the imaging modality. For example, paramagnetic contrast material may be used in MR, and iodinated radiographic contrast material is used for X-ray based modalities.

A common method of analyzing the images is to measure the tracer intensity profile in the main feeding artery as representative of the arterial input function (AIF). This analysis may be performed by manually selecting a portion of the image representing a region of the feeding artery and extracting the time-concentration-curve of the tracer at this region.

The AIF may be used when determining various tissue hemodynamic parameters quantitatively; for example, tissue blood volume, blood flow, transit time and bolus arrival time. These measurements depend on the specific features of the contrast agent injection, including the type and amount of contrast agent, and the injection rate.

### SUMMARY

In some embodiments, a method for operating a medical scanner comprises: injecting a radiopharmaceutical into a patient; performing one or more scans of the patient after the injecting using a medical scanner, each of the one or more scans including sampling an arterial input function of the patient at two or more locations in a same blood vessel of the patient at respectively different times for each location; and estimating the arterial input function of the patient based on the sampling, for use in medical imaging.

In some embodiments, a system for medical imaging comprises a scanner having a bed for receiving a patient and a plurality of detectors for detecting a radiopharmaceutical in a blood vessel of the patient. The bed or the plurality of detectors is movable. At least one processor is configured for: causing the scanner to perform one or more scans of the patient and detect emissions indicative of presence of the radiopharmaceutical in the blood vessel of the patent, each of the one or more scans including sampling an arterial input function of the patient at two or more locations in a same blood vessel at respectively different times for each location; and estimating the arterial input function of the patient based on the sampling, for use in medical imaging.

In some embodiments, a non-transitory, machine readable storage medium encoded with program instructions, such that when a processor executes the program instructions, the processor performs a method for: causing a scanner to perform one or more scans of a patient and detect emissions indicative of presence of a radiopharmaceutical in a blood vessel of the patient, each of the one or more scans including sampling an arterial input function of the patient at two or more locations in a same blood vessel at respectively different times for each location; and estimating the arterial input function of the patient based on the sampling, for use in medical imaging.

### BRIEF DESCRIPTION OF THE DRAWINGS

FIG. 1 is a schematic diagram of a system for controlling a medical scanner.

FIG. 2A shows a circulatory system of a patient, with a grid of image slices overlaid on the circulatory system.

FIG. 2B shows an estimated arterial input function (AIF) corresponding to samples collected at the three locations shown in FIG. 2A.

FIG. 3 is a diagram showing the regression estimate of AIF using one sample per scanning pass, and using multiple samples per scanning pass.

FIG. 4 is a diagram of a method of varying the interval between samples during multiple scanning passes.

FIGS. 5A and 5B show locations at which samples are collected, with each pass performed in the same direction.

FIG. 5C is a diagram of a method of varying the interval between samples during the scanning passes of FIGS. 5A-5B.

FIGS. 6A and 6B show locations at which samples are collected, with each pass performed in the opposite direction from the most recent previous pass.

FIG. 6C is a diagram of a method of varying the interval between samples during the scanning passes of FIGS. 6A-6B.

FIG. 7 is a flow chart of a method of estimating the AIF multiple samples per scanning pass.

FIG. 8 is a flow chart of the step of performing scanning passes in FIG. 7, in “shuttle mode”.

FIG. 9 is a flow chart of the step of performing scanning passes in FIG. 7, where the direction of motion (of the scanner or scanner bed) is the same during each pass.

### DETAILED DESCRIPTION

This description of the exemplary embodiments is intended to be read in connection with the accompanying drawings, which are to be considered part of the entire written description. In the description, relative terms such as “lower,” “upper,” “horizontal,” “vertical,”, “above,” “below,” “up,” “down,” “top” and “bottom” as well as derivative thereof (e.g., “horizontally,” “downwardly,” “upwardly,” etc.) should be construed to refer to the orientation as then described or as shown in the drawing under discussion. These relative terms are for convenience of description and do not require that the apparatus be constructed or operated in a particular orientation.

Systems described herein perform one or more scans of a patient using a medical scanner after injecting a radiopharmaceutical or contrast material. Each pass includes sampling an arterial input function (AIF) of the patient at two or more locations in the same blood vessel of the patient at respectively different times for each location. The AIF of the patient is estimated, based on the sampling, for use in medical imaging. The estimated AIF can be used for parametric positron emission tomography (PET) physiologically based pharmacokinetic (PBPK) modeling, for example. For example, the AIF is used for Patlak analysis, a compartment model technique that uses linear regression to identify and analyze pharmacokinetics of tracers involving irreversible uptake after the injection of a radiopaque or radioactive tracer (e.g., fluorodeoxyglucose). The Patlak model is used for the evaluation of nuclear medicine imaging data. The AIF and the Patlak model are used to determine the constants K and V₀, where K is the clearance determining the rate of entry of tracer into the peripheral (irreversible) compartment (e.g., an organ), and V₀ is the distribution volume of the tracer in the central (reversible) compartment (e.g., in a blood vessel).

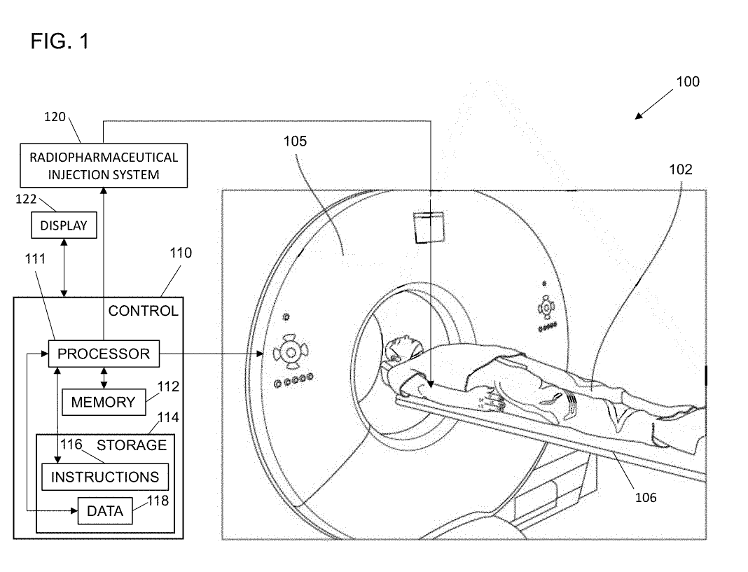

FIG. 1 shows a scanner system 100, including a control device 110 for controlling a scanner 105. The scanner 105 can be an magnetic resonance (MR) scanner, such as a “MAGNETOM VIDA” ™ scanner, a computed tomography (CT) scanner, such as a “SOMATOM CONFIDENCE RT Pro”™ CT Scanner, a PET scanner, such as the “BIOGRAPH HORIZON”™ PET/CT scanner, “SYMBIA INTEVO”™ Single-photon emission computed tomography (SPECT)/CT system, or an ultrasound scanner, such as the “ACUSON SC2000PRIME”™ cardiovascular ultrasound system, all sold by Siemens Medical Solutions USA, Inc. of Malvern, Pa. The scanner can include an automated contrast agent injection system 120 for automatic control of the injection profile, as provided by “CARE CONTRAST”™ in the “SOMATOM”™ scanner by Siemens Medical Solutions USA, Inc. of Malvern, Pa., where the contrast injector can be connected to the CT scanner, enabling synchronized injection and scanning. These are only examples, and other scanner makes and models may be used.

In some embodiments, the scanner 105 can be a continuous bed motion scanner, capable of moving a bed 106 of the scanner from a beginning of the one or more scans to an end of the one or more scans. The scanner 105 has a movable bed for receiving a patient and a plurality of detectors (not shown) for detecting a radiopharmaceutical in a blood vessel of the patient. Either the bed 106 or the plurality of detectors (not shown) are movable. In other embodiments, the scanner is capable of step-and-shoot scanning, with the sampling being performed at each of the two or more locations while the bed is not moving.

As discussed herein, a “scan” or “pass” can refer to a single translation by the scanner bed 106 with respect to the scanner 105, or a single translation by the scanner 105 with respect to the scanner bed 106. A scan or pass can proceed in a head-to-toe direction (corresponding to the bed moving in a direction from the patient's feet toward the patient's head), or a toe-to-head direction (corresponding to the bed moving in a direction from the patient's head toward the patient's feet). A scan or pass can refer to a complete pass (in which the patient's body from head to feet passes the scanner 105), or a partial pass (in which only a portion (less than 100%) of the patient's body (e.g., the patient's ear) passes the scanner 105). As used herein, the terms “scan” and “pass” can have any combination of these three attributes.

The control device 110 has a processor 111 configured to cause the scanner 105 to perform one or more scans of the patient and detect emissions indicative of presence of a the radiopharmaceutical in a the blood vessel of the patent. Each of the one or more scans includes sampling an arterial input function of the patient at two or more locations in a same blood vessel at respectively different times for each location. The processor 111 is further configured to estimate an arterial input function (AIF) of a radiopharmaceutical or contrast material, for physiologically based pharmacokinetic (PBPK) modeling of the subject based on the estimation.

The processor 111 is configured (e.g., by software) for controlling the scanner 105 based on the estimated AIF, injection profile, and delay between injecting the radiopharmaceutical or contrast agent and performing the scan. The processor 111 can issue commands to the automated injection system 120, to inject a selected dosage of radiopharmaceutical or contrast material in accordance with the estimated AIF. The processor 111 can have user input/output devices, such as a display 122, which can be a touch-screen capable of receiving user inputs and displaying outputs. Other input devices (e.g., keyboard or pointing device, not shown) may be included.

The processor 111 can include an embedded processor, a computer, a microcontroller, an application specific integrated circuit (ASIC), a programmable gate array, or the like. The control device 110 includes a main memory 112, which can include a non-transitory, machine readable storage medium such as dynamic random access memory (DRAM). The secondary memory comprises a non-transitory, machine readable storage medium 114, such as a solid-state drive, hard disk drive (HDD) and/or removable storage drive, which can include a solid state memory, an optical disk drive, a flash drive, a magnetic tape drive, or the like. The non-transitory, machine readable storage medium 114 can include tangibly store therein computer software instructions 116 for causing the scanner 105 to perform various operations (described herein) and data 118.

The injection system 120 can perform calibrated injections to patients, starting from a multi-dose solution of fluorodeoxyglucose (FDG), iodine, or other radiopharmaceuticals, or a contrast material. In some embodiments, the scanner 100 is not equipped with an automated injection system 120, in which case a separate injection system (not shown) may be used. For example, some systems can include an external injection system (not shown), such as the “IRIS™” Radiopharmaceutical Multidose Injector sold by Comecer S.p.A. of Castel Bolognese, Italy. In some embodiments, the injection system 120 has a wired or wireless communications link with the processor 111, for automatically transmitting dosage, injection protocol and scan delay to the injection system 120.

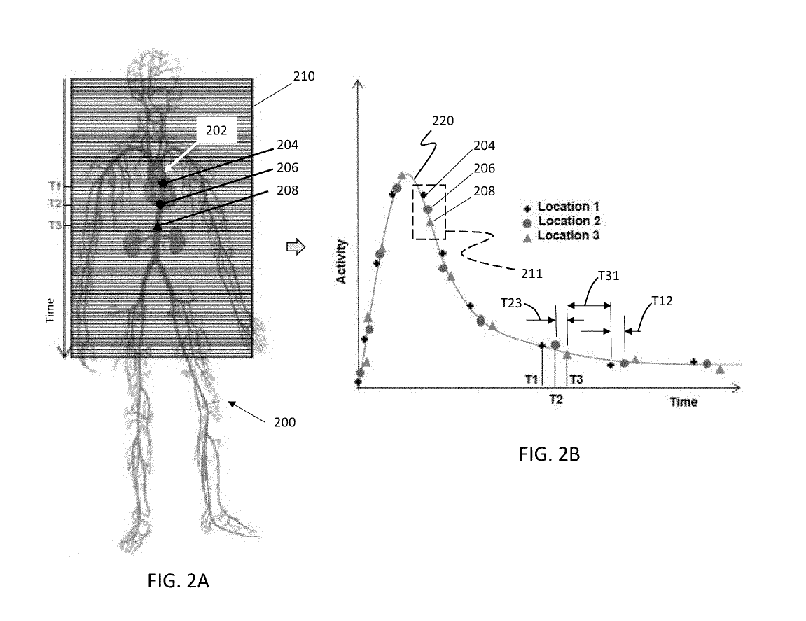

FIGS. 2A and 2B schematically show a method of estimating the AIF for medical imaging. FIG. 2A shows the vascular system 200 of a patient. A major blood vessel is selected for estimating the AIF. For example, in FIG. 2A, the aorta 202 is selected. The scanner 100 has a slice thickness (e.g., 2.0 mm), such that the scanner 100 is capable of collecting two slices separated by a distance greater than or equal to the slice thickness from each other. The grid 210 shows a grid of slices the scanner 100 can capture. The slice thickness (spatial resolution) correspond to resolution of the detectors (e.g., silicon photo-multipliers, SiPM, or photo-multipliers, PMT) in the scanner 105.

A plurality of samples are collected at respectively different times corresponding to respectively different slices. For example, in FIG. 2A, the cross shape at location 204 indicates a first sample collected at time T1. The circle 206 indicates a second sample collected at time T2. The triangle 208 indicates a third sample collected at time T3. The times T1-T3 are selected so that the corresponding locations 204, 206 and 208 are in three different slices. Although FIG. 2A shows three locations 204, 206, 208, the plurality of locations can include any number of locations greater than one and not greater than the number of slices that contain the blood vessel to be sampled. For example, in other embodiments, two or five locations can be sampled.

As shown in FIG. 2B, a set 211 of AIF sample data is collected, including a respective sample from each of the locations 204, 206, 208 in the vessel to be imaged. Each time another scanning pass is performed, another set 211 of AIF sample data is collected from each of the locations 204, 206, 208 in the same blood vessel. Thus, each set 211 of three sampling times T1-T3 in FIG. 2B corresponds to a respectively different pass.

In some embodiments, the time interval between successive samples in the same blood vessel can vary, and the temporal spacing between samples is non-uniform. For example, as shown in FIG. 2B, the time interval T31 between the last time activity data sample (at time T3) in one set 211 and the first activity data sample (at time T1) in the next set 211 can be different from the time interval T23 between the second and third sampling times, T2 and T3, respectively. In some embodiments, the time intervals between pairs of samples at successive locations within the same set 211 can be the same as each other. For example, the intervals T12 between successive samples at locations 204 and 206 can be the same as the interval T23 between samples at locations 206 and 208 within the same set 211. In the example of FIG. 2B, the intervals T12 and T23 within the same set 211 are relatively short intervals, compared to the relatively longer interval T31 between time T3 (at the end of one set 211) and T1 (at the beginning of the next set 211). In FIG. 2B, each set 211 begins with the cross (location 204 in the aorta) and ends with the triangle (location 208 in the aorta), corresponding to a head-to-toe, head-to-toe scanning pattern. Each pass includes moving the bed in the same direction between time T1 and T2, and between time T2 and T3, and then returning the bed to its initial position before the next pass. The length of time for returning the bed to its initial position is a reason the interval T31 is different from the interval T12 or T23. For example, in FIG. 2B, the interval T31 is longer than the interval T12 or T23. Although FIGS. 2A-2B show a head-to-toe, head-to-toe scanning pattern, the sampling can be performed when the scanner moves in the opposite direction, to form a toe-to-head, toe-to-head scanning pattern.

In some embodiments (not shown), all the intervals T12, T23 and T31 between successive samples can be a constant interval. In other embodiments (not shown), the interval T31 is shorter than the interval T12 or T23.

After a predetermined period (e.g., 3,600 seconds), the AIF can be estimated from the samples collected. For example, a polynomial regression can be performed to determine the activity (e.g., in mega-Becquerels/ml) as a function of time since injection.

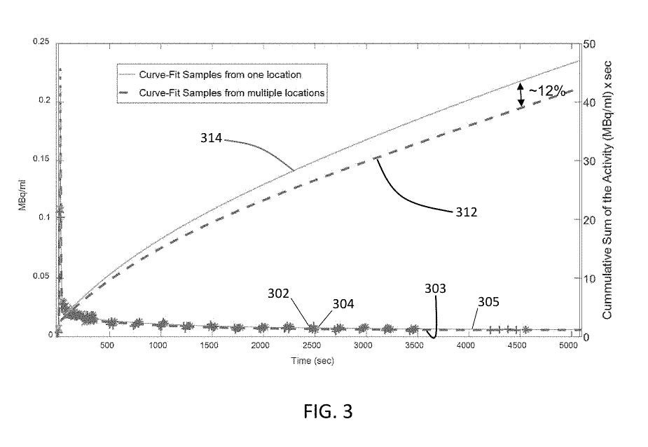

FIG. 3 shows the estimated AIF 303 based on samples 302 collected at multiple times/locations for each pass, juxtaposed with the estimated AIF 305 based on one sample 304 collected at a single time/location for each pass (“step-and-shoot”) during the period between 0 seconds and 360 seconds, and using continuous bed motion between 360 seconds and 5000 seconds. The estimated AIF 303 (based on samples 302 collected at multiple times/locations for each pass) begins with a lower value than the estimated AIF 305 based on one sample 304 collected at a single time/location for each pass. The estimated AIF 303 and the estimated AIF 305 both approach zero as the time since injection grows greater than one hour (3,600 seconds).

The accumulated activity curves 312 and 314 for the regression estimates show the impact of including multiple sampling times/locations for each pass. The accumulated activity curve 314 shows the total accumulated radiation per milliliter over time with a single sample per pass. The accumulated activity curve 312 shows the total accumulated radiation per milliliter over time with multiple samples per pass. As shown in FIG. 3, when multiple sample locations/times per pass are collected and used to estimate the AIF, the total accumulated radiation per ml is about 12% lower than in the case of a single sample per scan, due to the reduced influence of noise when multiple samples per pass are used.

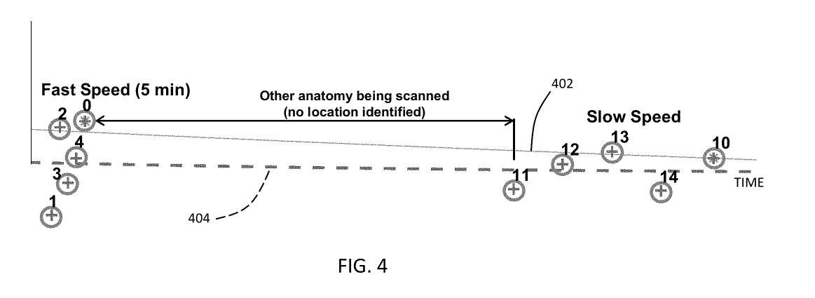

In some embodiments (e.g., as shown in FIG. 4), the speed of the bed motion can vary between passes. For example, the time-activity values 0-4 are collected during a first pass performed with a fast bed speed, and the time-activity values 10-14 are collected during a second pass performed with a slow bed speed. The time between pairs of successive samples 10-14 differs from the time between successive samples 0-4.

FIG. 4 schematically shows the impact of having greater temporal resolution in the AIF time-activity sample data. In FIG. 4, the points 0 and 10 represent samples collected using continuous bed motion, with a single sample per pass. The regression line 402 shows a portion of the estimated AIF corresponding to this time period. The points 0-4 and 10-14 represent samples collected using continuous bed motion with multiple times/locations per pass. The regression line 404 shows a portion of the estimated AIF corresponding to this time period using the higher temporal resolution. The estimated AIF 404 has smaller values than estimated AIF 402. Further, the time between samples 11-14 is different from the time between samples 1-4. Also, the time between sample 0 and sample 11 is different from any of the intervals within a single pass (from 0 to 4 or from 10 to 14).

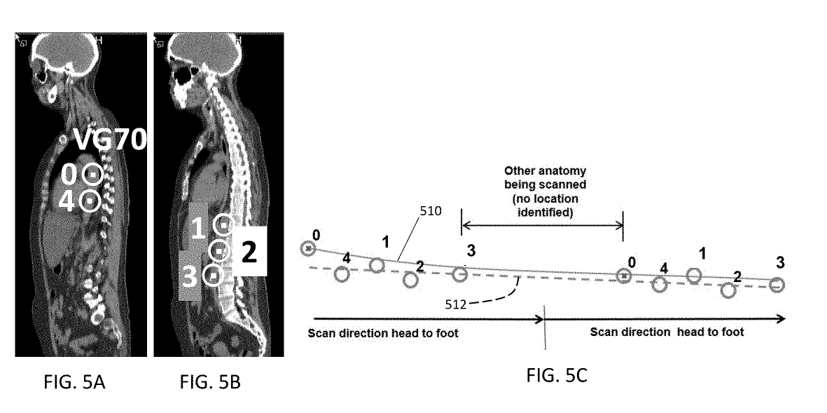

FIGS. 5A-5C show a set of scanning passes in which successive passes are in the same direction. For example, FIG. 5A shows samples 0 and 4 (head-to-foot) in a first pass, and FIG. 5B shows samples 1-3 (head-to-foot) in a second pass, with the bed returning to its initial position between passes. FIG. 5C shows the samples 0-1-2-3-4, 0-1-2-3-4 collected during two successive passes. As discussed above, the estimated AIF 512 using higher temporal sample density is lower than the estimated AIF 510 using a single sample per pass.

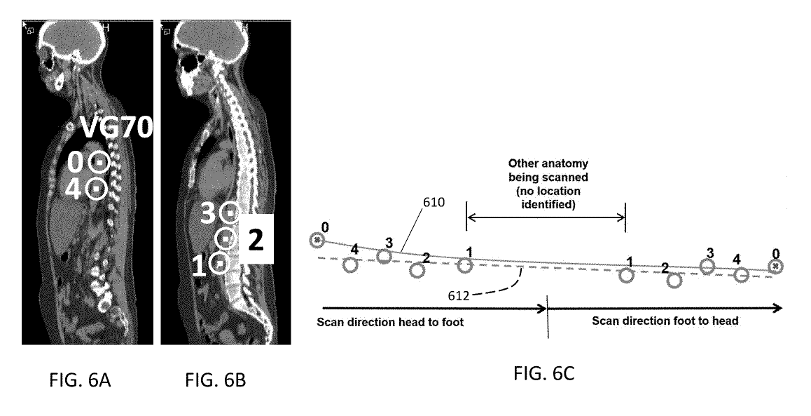

FIGS. 6A-6C show a set of scanning passes in which successive passes are in the opposite directions (also referred to as “shuttle mode”). For example, FIG. 6A shows samples 0 and 4 (head-to-foot) in a first pass, and FIG. 6B shows samples 1-3 (foot-to-head) in a second pass, without returning the bed to its initial position between passes. The direction of the second pass (FIG. 6B) is opposite the direction of the first pass (FIG. 6A). FIG. 6C shows the samples 0-1-2-3-4, 4-3-2-1-0 collected during two successive passes. As discussed above, the estimated AIF 612 using higher temporal sample density is lower than the estimated AIF 610 using a single sample per pass.

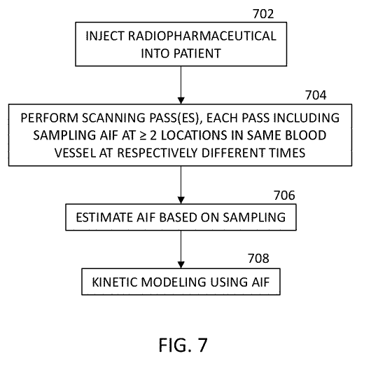

FIG. 7 is a flow chart of a method for operating a medical scanner 105.

At step 702, a radiopharmaceutical is injected into a patient. In some embodiments, the medical personnel manually inject the radiopharmaceutical into the patient's blood vessel, e.g., an arm blood vessel. In other embodiments, a radiopharmaceutical injection system 120 injects the radiopharmaceutical.

At step 704, the medical scanner 105 performs one or more scanning passes of the patient after the injecting. Each of the one or more scanning passes includes sampling an arterial input function of the patient at two or more locations in the same blood vessel of the patient at respectively different times for each location. The blood vessel is selected from among a plurality of blood vessels aligned with the direction of motion of the bed before performing the one or more scans. Because the selected blood vessel is aligned with the direction of motion of the bed, the scanner can sample the same blood vessel at respectively different locations and times during each pass. the two or more locations along the blood vessel are separated by at least the thickness of a slice the medical scanner can image

The medical scanner has a resolution determining a thickness of a slice the medical scanner can image. The sampling times are separated from each other by a sufficient time interval, so that each sample is collected from a different slice. For example, the scanner 105 may collect two, three, four, five, or more samples per pass. In some embodiments, each sample is collected as part of a different slice, and the number of samples is not greater than the number of slices.

At step 706, the processor 111 associated with the scanner 105 estimates the arterial input function (AIF) of the patient based on the sampling, for use in medical imaging.

At step 708, the scanner captures positron emission tomography (PET) sinogram data of the patient and performs PBPK modeling using the captured PET sinogram data. The PBPK model can be used for predicting the absorption, distribution, metabolism and excretion (ADME) of synthetic or natural chemical substances in the patient. The PBPK model may also be used in pharmaceutical research and drug development, and in health risk assessment for cosmetics or general chemicals. The PBPK model can be used to determine the correspondence between doses, exposure duration, and routes of administration.

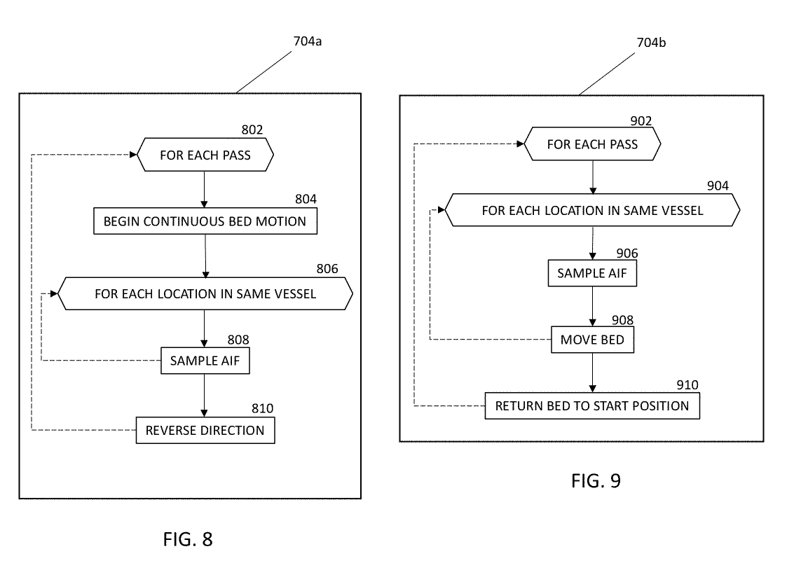

FIG. 8 is a flow chart of a continuous bed motion method 704a embodying step 704 of FIG. 7, in “shuttle” mode.

At step 802, a loop including steps 804-810 is performed once per scanning pass.

At step 804, the scanner begins a continuous bed motion for a partial pass encompassing an organ, or a complete pass.

At step 806, an inner loop including step 808 is performed for each location in the same artery at which the AIF is to be sampled.

At step 808 a sample is collected.

At step 810, at the completion of each pass, the scanner bed 106 (or scanner 105) reverses direction, so the next pass can be performed without first returning the scanner bed 106 to its original position.

FIG. 9 is a flow chart of a continuous bed motion method 704b embodying step 704 of FIG. 7, in “step and shoot” mode.

At step 902, a loop including steps 904-910 is performed once per scanning pass.

At step 904, an inner loop including steps 906-908 is performed for each location in the same artery at which the AIF is to be sampled.

At step 906, the scanner 105 collects a sample of AIF data.

At step 908 the scanner 105 moves the scanner bed 106 relative to the scanner 105 to collect the next sample. Alternatively, the scanner 105 itself moves relative to the scanner bed 106 to collect the next sample.

At step 910, at the completion of each pass, the scanner bed 106 (or scanner 105) returns to its original start position, so the next scanner pass can be performed in the same direction.

In some embodiments, a non-transitory, machine readable storage medium 114 can include tangibly store therein computer software instructions 116. When the processor 111 executes the program instructions 116, the processor 111 performs a method as shown in FIGS. 7 and 8, or FIGS. 7 and 9.

The methods and systems described herein provide improved accuracy in the arterial input function estimation and collect samples with greater temporal resolution without increasing the length of each scanning pass or the number of scanning passes.

The methods and system described herein may be at least partially embodied in the form of computer-implemented processes and apparatus for practicing those processes. The disclosed methods may also be at least partially embodied in the form of tangible, non-transitory machine readable storage media encoded with computer program code. The media may include, for example, RAMs, ROMs, CD-ROMs, DVD-ROMs, BD-ROMs, hard disk drives, flash memories, or any other non-transitory machine-readable storage medium, wherein, when the computer program code is loaded into and executed by a computer, the computer becomes an apparatus for practicing the method. The methods may also be at least partially embodied in the form of a computer into which computer program code is loaded and/or executed, such that, the computer becomes a special purpose computer for practicing the methods. When implemented on a general-purpose processor, the computer program code segments configure the processor to create specific logic circuits. The methods may alternatively be at least partially embodied in a digital signal processor formed of application specific integrated circuits for performing the methods.

Although the subject matter has been described in terms of exemplary embodiments, it is not limited thereto. Rather, the appended claims should be construed broadly, to include other variants and embodiments, which may be made by those skilled in the art.

What is claimed is:

1. A method for operating a medical scanner, comprising:
injecting a radiopharmaceutical into a patient;
performing one or more scans of the patient after the injecting using a medical scanner, each of the one or more scans including sampling an arterial input function of the patient at two or more locations in a same blood vessel of the patient at respectively different times for each location; and
estimating the arterial input function of the patient based on the sampling, for use in medical imaging.

2. The method of claim 1, wherein the scanner is a continuous bed motion scanner, and performing one or more scans includes moving a bed of the scanner from a beginning of the one or more scans to an end of the one or more scans.

3. The method of claim 2, wherein during a first one of the scans, the bed moves in a toe-to-head direction, and in a second one of the scans sequentially following the first one of the scans, the bed moves in a head-to-toe direction.

4. The method of claim 2, wherein during a first one of the scans, the bed moves in a head-to-toe direction, and in a second one of the scans sequentially following the first one of the scans, the bed moves in a toe-to-head direction.

5. The method of claim 2, wherein during a first one of the scans, the bed moves in a toe-to-head direction, and in a second one of the scans sequentially following the first one of the scans, the bed moves in the toe-to-head direction.

6. The method of claim 2, wherein during a first one of the scans, the bed moves in a head-to-toe direction, and in a second one of the scans sequentially following the first one of the scans, the bed moves in the head-to-toe direction.

7. The method of claim 2, further comprising selecting the blood vessel from among a plurality of blood vessels aligned with a direction of motion of the bed before performing the one or more scans.

8. The method of claim 1, wherein performing the one or more scans includes step-and-shoot scanning, with the sampling being performed at each of the two or more locations while the bed is not moving.

9. The method of claim 1, wherein the medical scanner has a resolution determining a thickness of a slice the medical scanner can image, and the two or more locations along the blood vessel are separated by at least the thickness of a slice the medical scanner can image.

10. The method of claim 1, wherein the samples of the arterial input function have non-uniform temporal spacing from each other.

11. The method of claim 1, further comprising:
capturing positron emission tomography (PET) sinogram data of the patient; and
performing kinetic modeling using the estimated arterial input function and the PET sinogram data.

12. A system for medical imaging, comprising:
a scanner having a bed for receiving a patient and a plurality of detectors for detecting a radiopharmaceutical in a blood vessel of the patient, the bed or the plurality of detectors being movable; and
at least one processor, configured for:
causing the scanner to perform one or more scans of the patient and detect emissions indicative of presence of the radiopharmaceutical in the blood vessel of the patent, each of the one or more scans including sampling an arterial input function of the patient at two or more locations in a same blood vessel at respectively different times for each location; and
estimating the arterial input function of the patient based on the sampling, for use in medical imaging.

13. The system of claim 12, wherein the bed is configured to move in a toe-to-head direction during a first one of the scans, and to move in a toe-to-head direction in a second one of the scans sequentially following the first one of the scans.

14. The system of claim 13, wherein the bed is configured to move in a toe-to-head direction during a first one of the scans, and to move in a toe-to-head direction in a second one of the scans sequentially following the first one of the scans.

15. The system of claim 12, wherein the scanner is configured for step-and-shoot scanning, with the sampling being performed at each of the two or more locations while the bed is not moving.

16. The system of claim 12, wherein the scanner has a resolution determining a thickness of a slice the scanner can image, and the two or more locations along the blood vessel are separated by at least the thickness of a slice the medical scanner can image.

17. The system of claim 12, wherein the scanner is configured for sampling the arterial input function with non-uniform temporal spacing from samples.

18. The system of claim 12, wherein the scanner is a positron emission tomography scanner.

19. A non-transitory, machine readable storage medium encoded with program instructions, such that when a processor executes the program instructions, the processor performs a method for:
causing a scanner to perform one or more scans of a patient and detect emissions indicative of presence of a radiopharmaceutical in a blood vessel of the patient, each of the one or more scans including sampling an arterial input function of the patient at two or more locations in a same blood vessel at respectively different times for each location; and
estimating the arterial input function of the patient based on the sampling, for use in medical imaging.

20. The non-transitory, machine readable storage medium of claim 19, wherein the scanner is a continuous bed motion scanner configured for detecting the emissions while moving a bed of the scanner from a beginning of the one or more scans to an end of the one or more scans.

21. The non-transitory, machine readable storage medium of claim 19, wherein the samples of the arterial input function have non-uniform temporal spacing from each other.
